(19) United States
(12) Patent Application Publication
(10) Pub. No.: US 2024/0408164 A1
(43) Pub. Date: Dec. 12, 2024

(54) COMPOSITION COMPRISING PRUNE FOR PREVENTING OR RELIEVING LOW ANTERIOR RESECTION SYNDROME

(71) Applicant: SAMAUNG LIFE PUBLIC WELFARE FOUNDATION, Seoul (KR)

(72) Inventors: Yong Beom CHO, Seongnam-si (KR); Yoon Ah PARK, Seoul (KR); Woo Yong LEE, Seoul (KR); Seong Hyeon YUN, Seoul (KR); Hee Cheol KIM, Seoul (KR); Jung Wook HUH, Seoul (KR); Jung Kyong SHIN, Seoul (KR); Dae Hee PYO, Seoul (KR); Park Jee HYUN, Seoul (KR)

(21) Appl. No.: 18/697,585

(22) PCT Filed: Aug. 19, 2022
(86) PCT No.: PCT/KR2022/012394

§ 371 Date: May 23, 2024

(30) Foreign Application Priority Data
Oct. 1, 2021 (KR) 10-2021-0130664

(51) Int. Cl. A61K 36/736; A61P 1/00

(52) U.S. Cl. CPC A61K 36/736; A61P 1/00

(57) ABSTRACT

The present invention relates to a food composition or health functional food containing prune. Consumption of the food composition or health functional food has the effect of preventing and alleviating low anterior resection syndrome.

### TECHNICAL FIELD

The present invention relates to a composition for preventing or alleviating low anterior resection syndrome containing prune.

### BACKGROUND ART

Low anterior resection syndrome (hereinafter referred to as “LARS”) is a change experienced by 90% of patients after rectal resection, and means that defecation-related functions are changed after surgery although the severity of symptoms varies. In the case of changed defecation-related functions, overlapping symptoms occur, such as frequent defecation, urgency, feeling of residual stool, stool leakage, and fecal incontinence, and bowel fragmentation also occurs in which there is a continuous feeling of urgency to defecate while having several small bowel movements within 1 to 2 hours (Eun Lee, A Phenomenological Study on the Disease Experience of Rectal Cancer Patients Who Underwent Low Anterior Resection, 2015) The specific physiological mechanism of LARS has not yet been revealed, but the causes thereof are presumed to be a decrease in rectal volume and compliance, damage to the autonomic nervous system, damage to the sphincter due to insertion of an automatic anastomosis device, a decrease in the sampling reflex that distinguishes the form of stool, and the like (Romanisyn et al, 2012).

This low anterior resection syndrome has been reported to be a major factor impairing the quality of life of rectal cancer patients (Eun Lee and Keum-Soon Kim, 2014; Vironen, Kairaluoma, Aalto & Kellokumpu, 2006), but studies thereon are still insufficient.

### DISCLOSURE

### Technical Problem

One aspect is to provide a food composition for preventing or alleviating low anterior resection syndrome containing prune.

Another aspect is to provide a health functional food for preventing or alleviating low anterior resection syndrome containing prune.

### Technical Solution

One aspect provides a food composition for preventing or alleviating low anterior resection syndrome containing prune.

The prune refers to dried plums, and may also be referred to as “dried plums”.

According to one embodiment, the amount of the prune may be 60 g to 100 g, without being limited thereto.

According to one embodiment, the low anterior resection syndrome may occur in an individual who has undergone radical resection or low anterior resection.

The radical resection may refer to surgery that completely removes a structure or organ for the purpose of completely curing the disease.

The low anterior resection may be a radical surgery that may be performed when the shortest distance from the cancer in the rectum to the anal verge is 5 cm to 15 cm.

According to one embodiment, the individual may be an individual diagnosed with cancer.

According to one embodiment, the cancer may be rectal cancer or rectosigmoid colon cancer.

The term “rectal cancer” may mean cancer occurring in the rectum, and the term “rectosigmoid colon cancer” may mean cancer occurring in the lower part of the sigmoid colon and the upper part of the rectum.

The cancer may be primary, and the term “primary” means the disease itself that is not caused by another factor, and the term “primary rectal cancer” may refer to rectal cancer that has not metastasized from another cancer.

The term “low anterior resection syndrome” may mean that overlapping symptoms such as frequent defecation, urgency, feeling of residual stool, stool leakage, and fecal incontinence occur after rectal resection. The low anterior resection syndrome may show bowel fragmentation and symptoms in which there is a continuous feeling of urgency to defecate while having several small bowel movements within 1 to 2 hours.

The term “prevention” refers to any action that delays or suppresses the occurrence of low anterior resection syndrome after surgery.

The term “alleviation” refers to any action that alleviates or ameliorates low anterior resection syndrome after surgery.

Examples of the food composition include, but are not limited to, various foods, beverages, gum, tea, vitamin complexes, etc.

Foods for preventing or alleviating low anterior resection syndrome according to the present invention are in the form of tablets, capsules, pills, liquids, or the like.

Other ingredients are not particularly limited, except that the food composition for preventing or alleviating low anterior resection syndrome according to the present invention contains the above-mentioned prune as an essential ingredient. The food composition may further contain various flavoring agents, natural carbohydrates, and the like, like a conventional beverage, as additional ingredients.

Examples of the natural carbohydrates include conventional sugars, such as monosaccharides (e.g., glucose, fructose, etc.), disaccharides (e.g., maltose, sucrose, etc.), and polysaccharides (e.g., dextrin, cyclodextrin, etc.); and sugar alcohols such as xylitol, sorbitol, erythritol or the like. In addition to those described above, flavoring agents may advantageously be used, such as a natural flavoring agent (thaumatin, stevia extract (e.g., rebaudioside A, glycyrrhizin, etc.)) and a synthetic flavoring agent (saccharin, aspartame, etc.). The natural carbohydrate is generally used in an amount of about 1 to 20 g, preferably about 5 to 12 g, based on 100 ml of the composition of the present invention.

In addition, the composition of the present invention may contain various nutrients, vitamins, minerals (electrolytes), flavoring agents such as synthetic flavoring agents and natural flavoring agents, colorants, fillers (cheese, chocolate, etc.), pectic acid and its salt, alginic acid and its salt, organic acids, protective colloidal thickeners, pH adjusting agents, stabilizers, preservatives, glycerin, alcohol, carbonizing agents that are used in carbonated beverages, etc.

Another aspect provides a health functional food for preventing or alleviating low anterior resection syndrome containing prune.

The contents regarding the prune, low anterior resection, low anterior resection syndrome, and prevention and alleviation are the same as those described above.

As used in the present invention, the term “health functional food” refers to foods manufactured and processed in the form of tablets, capsules, powders, granules, liquids, and pills using raw materials or ingredients that have functionality beneficial for the human body. Here, the term “functionality” means controlling nutriments on the structure and function of the human body or achieving useful effects on health such as physiological effects. The health functional food of the present invention may be manufactured by a method commonly used in the art, and may be manufactured by adding raw materials and ingredients commonly added in the art during manufacture. In addition, the formulation of the health functional food may also be manufactured without limitation as long as it is a formulation recognized as a health functional food. The food composition of the present invention may be manufactured in various forms, and has the advantage of having no side effects that may occur when taking drugs for a long period of time as it contains food as a raw material, unlike general drugs. In addition, the health functional food of the present invention has excellent portability. Thus, the health functional food of the present invention may be consumed as a supplement to alleviate low anterior resection syndrome.

There is no limit to the form that the health functional food of the present invention can take, and the term “health functional food” may include all foods in a conventional sense, and may be used interchangeably with terms known in the art, such as functional food. In addition, the health functional food containing prune according to the present invention may be prepared by mixing prune with known additives and other appropriate auxiliary ingredients that may be contained in foods according to the selection of a person skilled in the art. Examples of foods to which the health functional food of the present invention may be added include meat, sausages, bread, chocolate, candies, snacks, confectionery, pizza, ramen, other noodles, gum, dairy products, including ice cream, various soups, beverages, teas, drinks, beverages, and vitamin complexes. Juices, teas, jellies, etc. may be prepared using prune according to the present invention as a main ingredient. The term “food” also includes food that is used as feed for animals.

### Advantageous Effects

Taking the composition containing prune according to the present invention has the effect of preventing and alleviating low anterior resection syndrome.

### BRIEF DESCRIPTION OF DRAWINGS

FIG. 1 is a table schematically showing an experimental design for evaluating the effect of prune on low anterior resection syndrome.

FIG. 2 depicts data showing the results of evaluating global health status, physical function, role function, emotional function, cognitive function, and social function in a prune group and a no-prune group through EORTC-QLQ 30 questionnaire.

FIG. 3 depicts data showing the results of evaluating the fatigue, nausea, pain, dyspnea, constipation, diarrhea, sleep disturbance, and appetite loss in a prune group and a no-prune group through EORTC-QLQ 30 questionnaire.

### BEST MODE

One aspect provides a food composition for preventing or alleviating low anterior resection syndrome containing prune.

According to one embodiment, the low anterior resection syndrome may occur in an individual who has undergone radical anterior resection or low anterior resection.

According to one embodiment, the individual may be an individual diagnosed with cancer.

According to one embodiment, the cancer may be rectal cancer or rectosigmoid colon cancer.

Another aspect provides a health functional food for preventing or alleviating low anterior resection syndrome containing prune.

### MODE FOR INVENTION

One or more embodiments will be described in more detail by way of examples. However, these examples are intended to illustrate one or more embodiments and the scope of the present invention is not limited to these examples.

### Experimental Example 1. Study Design and Method

### 1.1. Study Purpose and Conducting Method

To evaluate the effect of prune on low anterior resection syndrome, the study was conducted at a single institution and was a double-arm, nonblinded, randomized, controlled trial.

### 1.2. Blinding

Because a treatment equivalent to a placebo for prune could not be designed, it was impossible to conduct a double-blind experiment due to the nature of the study. Therefore, blinding was applied only to the remaining researchers, excluding the researcher who explained prune intake to patients assigned to the prune group, and to the group assignment of patients other than the patient himself/herself.

### 1.3. Selection of Participants

Among patients (older than 18 years but younger than 70 years of age) who diagnosed with primary rectal cancer or rectosigmoid colon cancer from August 2019 to March 2021, patients who underwent radical resection or low anterior resection were selected as participants. However, the following patients who met the above condition were excluded from the study: patients who underwent preoperative anticancer radiotherapy; patients who underwent colostomy; patients who underwent combined resection such as Cholecystectomy, hepatectomy, and lobectomy; patients who underwent preoperative endoscopic stent insertion; patients who refused to participate in the study; patients who had a history of surgery for rectosigmoid colon cancer or rectal cancer in the past; and patients who had a history of bowel resection for any reason in the past. In addition, patients with diabetes mellitus, chronic constipation, or food or fruit allergy were also excluded due to safety concerns. This study adhered to the Declaration of Helsinki and was reviewed and approved by the Ethics Committee of the Institutional Review Boards in Samsung Medical Center (2019-06-104). Written informed consent was also obtained from all study participants. This clinical trial was registered with the Clinical Research Information Service, which is a Korean registration system for clinical trials (KCT0006085).

### 1.4. Participant Group Randomization

To design a study with a 20% or greater minimal predefined reduction in the incidence of LARS between the prune group and the no-prune group, at least 66 patients in each study group were required to achieve a power greater than 80% (β) with a type I error rate of 5% (α).

Patients were allocated randomly to the prune group or no-prune group by computer-based randomization. Once the inclusion criteria were met, patients were randomized to one of these two groups with a 1:1 allocation ratio.

### 1.5. Treatment According to Participant Group

The prune group was instructed to consume a total of 60 g of prune twice (in the morning and afternoon beginning) for 3 days starting from the first day of solid food diet. If there were no symptoms corresponding to the stability evaluation variables (stabbing severe lower abdominal pain, diarrhea, bloody stool, fever, chills, etc.), a total of 100 g of prunes was consumed twice in the morning and afternoon. In this study, “Taylor Prunes” grown in California, USA, produced by Taylor Brothers Farms, Inc. and imported into Korea were used.

The patients in the untreated control group, which was the no-prune group as an untreated control group, followed a normal diet without consuming any product containing dried plums during the study period. However, consumption of health supplement foods containing lactic acid bacteria or probiotics and other health supplement foods having the effect of alleviating constipation or improving gastrointestinal function was restricted.

### 1.6. Outcomes

To derive the main results of this study, the incidence of LARS was determined at the first visit to the outpatient clinic after surgery by the Korean version of a LARS questionnaire, and the score of quality of life defined at the first visit after surgery was assessed using the Korean version of the European Organization for Research and Treatment of Cancer's Quality of Life Questionnaire 30 (EORTC-QLQ30). The incidence of low anterior resection syndrome (hereinafter referred to as “LARS”) was determined at the first visit to the outpatient clinic after surgery by a questionnaire containing the five items (1 to 5) shown in Table 1 below. The points for each question were applied as shown in Table 1 below. Scores of 20 points or less were said to indicate “no LARS,” while those of 20 points to 29 points were defined as suggestive of “minor LARS.” Scores of 30 points to 42 points were indicative of the presence of “major LARS.” Quality of life was assessed using the Korean version of the EORTC QLQ-C30, version 3.0 shown in Table 2 below.

TABLE 1

| | |
|---|---|
| 1. Do you sometimes find | 1) No, not at all. (0 points) |
| it difficult to control | 2) Yes, but only once a week or not at |
| your farts? | all. (4 points) |
| | 3) Yes, sometimes it is difficult to |
| | control more than once a week. |
| | (7 points) |
| 2. Have you ever | 1) No, not at all. (0 points) |
| experienced symptoms of | 2) Yes, but only once a week or not at |
| watery stool? | all. (3 points) |
| | 3) Yes, sometimes it is difficult to |
| | control more than once a week. |
| | (3 points) |
| 3. How often do you | 1) More than 7 times in 24 hours a day |
| visit the bathroom to | (4 points) |
| defecate? (feces, not | 2) 4 to 7 times in 24 hours a day |
| urine) | (2 points) |
| | 3) 1 to 3 times in 24 hours a day |
| | (0 points) |
| | 4) It is difficult to visit once in 24 |
| | hours a day. (5 points) |
| 4. Have you ever gone to | 1) No, not at all. (0 points) |
| the bathroom again to | 2) Yes, but only once a week or not at |
| have a bowel movement | all. (9 points) |
| within an hour since | 3) Yes, sometimes it is difficult to |
| your last bowel | control more than once a week. |
| movement? | (11 points) |
| 5. Have you ever felt | 1) No, not at all. (0 points) |
| the urge to defecate so | 2) Yes, but only once a week or not at |
| much that you had to | all. (11 points) |
| rush to the bathroom? | 3) Yes, sometimes it is difficult to |
| | control more than once a week. |
| | (16 points) |
| 6. To what extent do you | 1) Bowel function does not affect |
| think your “quality of | “quality of life” at all. |
| life” is reduced due to | 2) Bowel function has a slight impact |
| your bowel function? | on “ ‘quality of life.” |
| (This question is | 3) Bowel function affects “quality of |
| excluded from LARS | life” and has some impact on daily |
| diagnosis) | life. |
| | 4) Bowel function has a significant |
| | impact on “quality of life” and has a |
| | significant impact on daily life. |

TABLE 2

| | | | Almost | |
|---|---|---|---|---|
| | Never | Sometimes | always | Always |
| 1. Do you have trouble doing | 1 | 2 | 3 | 4 |
| tasks that require strength, | | | | |
| such as carrying heavy | | | | |
| shopping bags or bags? | | | | |
| 2. Do you find it difficult to | 1 | 2 | 3 | 4 |
| walk for long periods of time? | | | | |
| 3. Do you find it difficult to | 1 | 2 | 3 | 4 |
| take short walks outside your | | | | |
| home? | | | | |
| 4. Do you prefer to lie down or | 1 | 2 | 3 | 4 |
| recline in a chair during the day? | | | | |
| 5. Do you need help while | 1 | 2 | 3 | 4 |
| eating, dressing, washing up, | | | | |
| or using the bathroom? | | | | |

* Please answer based on the past week.

| | | | | |
|---|---|---|---|---|
| 6. Have you ever felt limited | 1 | 2 | 3 | 4 |
| in your ability to work or | | | | |
| carry out other daily activities? | | | | |
| 7. Have you ever felt limited | 1 | 2 | 3 | 4 |
| in your hobbies or leisure | | | | |
| activities? | | | | |
| 8. Have you ever felt short of | 1 | 2 | 3 | 4 |
| breath? | | | | |
| 9. Have you ever felt pain? | 1 | 2 | 3 | 4 |
| 10. Have you ever thought you | 1 | 2 | 3 | 4 |
| needed a break? | | | | |
| 11. Have you ever had trouble | 1 | 2 | 3 | 4 |
| getting a good night's sleep? | | | | |
| 12. Have you ever felt unwell? | 1 | 2 | 3 | 4 |
| 13. Have you lost your appetite? | 1 | 2 | 3 | 4 |
| 14. Have you ever felt nauseous? | 1 | 2 | 3 | 4 |
| 15. Have you ever vomited? | 1 | 2 | 3 | 4 |

* Please answer based on the past week.

| | | | | |
|---|---|---|---|---|
| 16. Have you ever experienced | 1 | 2 | 3 | 4 |
| symptoms of constipation? | | | | |
| 17. Have you ever had diarrhea? | 1 | 2 | 3 | 4 |
| 18. Did you feel tired? | 1 | 2 | 3 | 4 |
| 19. Have you ever experienced | 1 | 2 | 3 | 4 |
| difficulty in carrying out | | | | |
| your daily life due to pain? | | | | |
| 20. Have you ever had trouble | 1 | 2 | 3 | 4 |
| concentrating when reading the | | | | |
| newspaper or watching television? | | | | |
| 21. Did you feel nervous? | 1 | 2 | 3 | 4 |
| 22. Are you suffering from | 1 | 2 | 3 | 4 |
| anxiety? | | | | |
| 23. Did you feel irritated? | 1 | 2 | 3 | 4 |
| 24. Have you felt depressed? | 1 | 2 | 3 | 4 |
| 25. Have you noticed memory | 1 | 2 | 3 | 4 |
| loss? | | | | |
| 26. Has your health condition | 1 | 2 | 3 | 4 |
| or medication caused any | | | | |
| difficulties in your home life? | | | | |
| 27. Has your health condition | 1 | 2 | 3 | 4 |
| or medication caused any | | | | |
| difficulties in your social life? | | | | |
| 28. Have you experienced | 1 | 2 | 3 | 4 |
| financial difficulties due to | | | | |
| your health condition or | | | | |
| medication? | | | | |

* Please read the following questions and circle the number
which best corresponds to what you think, among numbers 1 to 7.
29. How would you rate your overall health over the past week?

| | | | | | | |
|---|---|---|---|---|---|---|
| 1 very bad | 2 | 3 | 4 | 5 | 6 | 7 very good |

30. How would you rate your overall quality of life over the past week?

| | | | | | | |
|---|---|---|---|---|---|---|
| 1 very bad | 2 | 3 | 4 | 5 | 6 | 7 very good |

### 1.7. Statistical Analysis

Considering the patients who completed the study, per-protocol analysis was performed throughout the analysis period. Comparisons of continuous and categorical variables were performed using Student's t-test and Chi-square test. To define the independent risk or protective factors related to the incidence of LARS, univariable and multivariable regression analyses were performed. The variables significantly correlated with the incidence of LARS in the univariable regression analysis were reconsidered as explanatory variables in multivariable regression analysis. In all cases, p-values<less than 0.05 were considered statistically significant. All statistical analyses were performed using R version 3.6.1 (R Foundation for Statistical Computing, Vienna, Austria).

### Example 1. Experimental Results

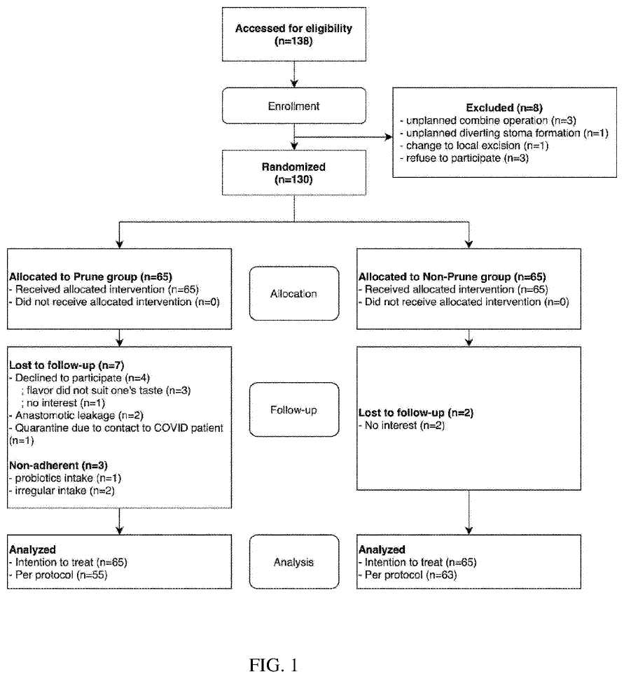

As shown in FIG. 1, during the study period, 138 patients were selected for clinical trials. Among these patients, three who underwent combined resection, one who underwent diverting stoma, one who underwent local excision other than primary rectal anastomosis, and three who refused to participate in clinical trials were excluded. Consequently, 130 patients participated and were randomized to the prune group and the no-prune groups (n=65 patients per group). Among the patients allocated to the prune group, a total of 10 patients dropped out, including four who declined to consume prunes in the middle of the study period, two who underwent emergent stoma formation due to anastomotic leakage, one who was lost due to self-quarantine after experiencing close contact with patient confirmed to have coronavirus disease 2019, and three who violated the study protocols, such as probiotic intake and irregular prune intake. Among the patients allocated to the no-prune group, two dropped out during the study. Finally, 55 patients in the prune group and 63 patients in the no-prune group were selected for analysis.

TABLE 3

| | Prune | No prune | |
|---|---|---|---|
| | (n = 55) | (n = 63) | P-value |
| Age, n (%), years | | | 0.648 |
| ≥65 | 7 (12.7) | 11 (17.5) | |
| <65 | 48 (87.3) | 52 (82.5) | |
| Sex, n (%) | | | 0.487 |
| Male | 40 (72.7) | 41 (65.1) | |
| Female | 15 (27.3) | 22 (34.9) | |
| BMI, n (%) | | | 0.722 |
| ≥25 kg/m² | 21 (38.2) | 21 (33.3) | |
| <25 kg/m² | 34 (61.8) | 42 (66.7) | |
| CEA, n (%) | | | 1.000 |
| ≥5 ng/ml | 3 (5.5) | 4 (6.3) | |
| <5 ng/ml | 52 (94.5) | 59 (93.7) | |
| Tumor location, n (%) | | | 0.526 |
| Sigmoid | 8 (14.5) | 12 (19.0) | |
| Rectosigmoid | 20 (36.4) | 17 (27.0) | |
| Rectum | 27 (49.1) | 34 (54.0) | |
| Surgery type, n (%) | | | 0.442 |
| Laparoscopic | 52 (94.5) | 56 (88.9) | |
| Robotic | 3 (5.5) | 7 (11.1) | |
| Name of surgery, n (%) | | | 0.915 |
| LAR | 51 (92.7) | 57 (90.5) | |
| AR | 4 (7.3) | 6 (9.5) | |
| pT stage, n (%) | | | 0.964 |
| pT1 | 6 (10.9) | 7 (11.1) | |
| pT2 | 21 (38.1) | 26 (41.3) | |
| pT3 | 28 (51.0) | 30 (47.6) | |
| pN stage, n (%) | | | 0.454 |
| pN0 | 49 (89.1) | 52 (82.5) | |
| pN1 | 6 (10.9) | 11 (17.5) | |

BMI, body mass index;
CEA, carcinoembryonic antigen;
LAR, low anterior resection;
AR, anterior resection

In Table 3 above, the baseline characteristics (age, sex, surgical site, tumor location, type of surgery, degree of cancer progression, etc.) of the two groups were compared. The pT stage is classified according to the depth at which the tumor has invaded the intestinal wall, and the pN stage is determined according to the degree of lymph node metastasis. As shown in Table 3 above, as a result of the comparison, no significant differences in the clinicopathologic characteristics with respect to the baseline characteristics were observed. In addition, as shown in Table 4 below, the operative characteristics and postoperative outcomes of the two groups of patients were compared, and there were no differences in operating time or length of hospital stay between the two groups. There was no significant difference in the confirmation rate of ileus, a common postoperative complication.

TABLE 4

| | Prune | No prune | |
|---|---|---|---|
| Variables, median (IQR) | (n = 55) | (n = 63) | P-value |
| Operation time, min | 163 (135-200) | 169 (138-210) | 0.405 |
| EBL, mL | 50 (30-130) | 50 (30-100) | 0.614 |
| Hospital stay, days | 8 (7-9) | 8 (7-9) | 1.000 |
| Bowel movement, days | 3 (3-4) | 3 (3-5) | 0.812 |
| First solid diet, days | 4 (4-5) | 4 (4-5) | 0.912 |
| Postoperative morbidity, | 6 (10.9) | 7 (11.1) | 1.000 |
| n (%) | | | |
| Wound | 0 | 1 (1.6) | — |
| Ileus | 4 (7.3) | 5 (7.9) | 1.000 |
| Urinary retention | 2 (3.6) | 1 (1.6) | — |

IQR, interquartile range;
EBL, estimated blood loss.

The LARS questionnaire results are presented in Table 5 below. There was a significant difference in the total incidence of LARS, including the rates of both major and minor LARS, between the two groups. In addition, when patients with LARS were analyzed, the incidence of major LARS was significantly lower in the prune group compared to the no-prune group (27.3% vs. 76.2%). In addition, it was confirmed that the proportions of LARS (hereinafter referred to as “major LARS”) indicating the degree of severity were 33.3% and 81.2% in the prune group and no-prune group, respectively, indicating that the major LARS in the prune group was significantly lower.

TABLE 5

| | Prune | No prunes | |
|---|---|---|---|
| | (n = 55) | (n = 63) | P-value |
| Incidence of LARS | | | <0.001 |
| LARS (+) | 15 (27.3) | 48 (76.2) | |
| LARS (−) | 40 (72.7) | 15 (23.8) | |
| Severity of LARS | | | 0.001 |
| Major LARS | 5 (33.3) | 39 (81.2) | |
| Minor LARS | 10 (67.7) | 9 (18.8) | |

LARS; low anterior resection syndrome.

When comparative analysis for each item composing the LARS questionnaire was performed, findings for the number of toilet visits, revisiting the toilet within one hour, and urgency symptoms exhibited significant differences between the groups as shown in Table 6 below. Gas continence and watery stool symptoms did not show statistical differences, but frequency, toilet re-visit within 1 hour (<1 hr re-toilet), and urgency showed significant differences between the prune group and the no-prune group. This indicates that prune intake alleviates LARS symptoms.

TABLE 6

| | Prune | No prunes | |
|---|---|---|---|
| LARS component | (n = 55) | (n = 63) | P-value |
| Gas control (max 7) | 3.6 (0.4) | 3.8 (0.9) | 0.551 |
| Watery stool (max 3) | 2.1 (1.9) | 2.2 (1.2) | 0.339 |
| Frequency (max 5) | 2.5 (0.5) | 4.2 (1.8) | 0.034 |
| <1-hour re-toilet | 6.3 (2.5) | 9.3 (3.8) | 0.041 |
| (max 11) | | | |
| Urgency (max 16) | 8.2 (6.9) | 13.2 (7.0) | 0.017 |

In addition, as shown in Table 7 below, in univariable regression analysis for the incidence of LARS, male sex, tumor location at the rectum, anastomosis level below the peritoneal reflexion, and prune intake were significantly correlated factors. In multivariable regression analysis, tumor location at the rectum and prune intake were confirmed as independent risk and protective factors, respectively. The odds ratio (95% confidence interval) of prune intake was 0.38 (0.10-0.89).

TABLE 7

| | Univariate analysis | | Multivariate analysis | |
|---|---|---|---|---|
| | OR (95% CI) | P-value | OR (95% CI) | P-value |
| Age ≥ 65 years | 2.21 (0.81-5.98) | 0.183 | | |
| Male | 3.04 (1.27-7.23) | 0.021 | 2.95 (0.95-5.32) | 0.064 |
| BMI ≥ 25 kg/m² | 1.47 (0.69-3.16) | 0.418 | | |
| CEA ≥ 5 ng/ml | 3.81 (0.71-20.5) | 0.212 | | |
| Tumor location | | | | |
| Rectosigmoid vs. | 1.41 (0.25-8.00) | 0.891 | | |
| sigmoid | | | | |
| Rectum vs. sigmoid | 19.9 (4.19-94.5) | <0.001 | 14.3 (3.10-32.1) | <0.001 |
| Surgery type | | | | |
| Robotic vs. | 0.93 (0.25-3.40) | 0.903 | | |
| laparoscopic | | | | |
| Surgery name | | | | |
| LAR vs. AR | 7.2 (0.88-58.8) | 0.081 | 5.29 (0.75-25.6) | 0.213 |
| pT stage | | | | |
| T2 vs. T1 | 1.19 (0.34-4.17) | 0.789 | | |
| T3 vs. T1 | 1.13 (0.33-3.88) | 0.792 | | |
| pN stage | | | | |
| N1 vs. N0 | 1.30 (0.46-3.65) | 0.814 | | |
| Prune | 0.14 (0.06-0.32) | <0.001 | 0.38 (0.10-0.89) | 0.018 |

OR, odds ratio;
CI, confidence interval;
BMI, body mass index;
CEA, carcinoembryonic antigen;
LAR, low anterior resection;
AR, anterior resection

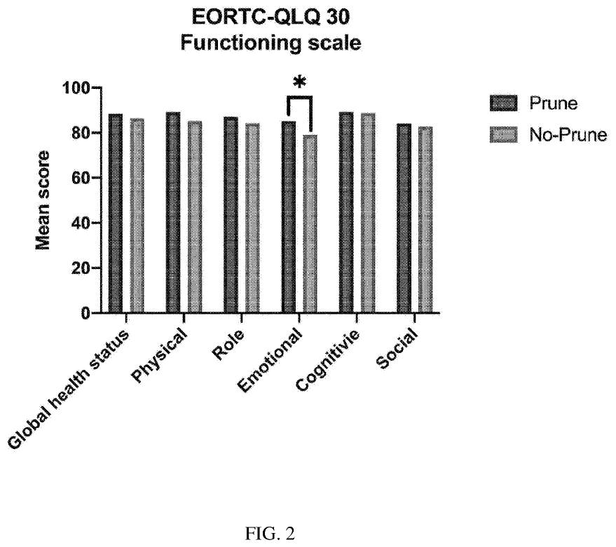

As a result of analysis of the EORTC-QLQ 30 results, as shown in FIG. 2, the prune group and the no-prune group showed similar levels for global health status, role function, cognitive function, and social function, but the prune group showed higher scores in physical function and emotional function than the no-prune group.

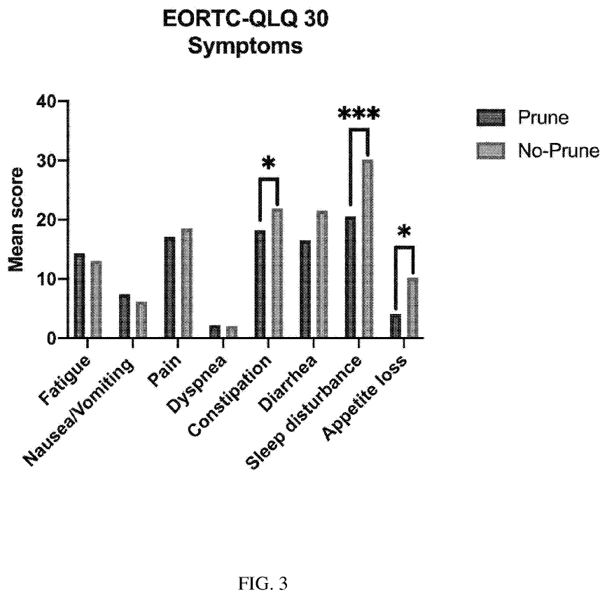

In addition, as shown in FIG. 3, the prune group showed lower scores for constipation, diarrhea, sleep disturbance, and appetite loss in the symptom domain than the no-prune group, indicating that prune intake significantly improves the general quality of life.

1. A food composition for preventing or alleviating low anterior resection syndrome containing prune.

2. The food composition of claim 1, wherein the low anterior resection syndrome occurs in an individual who has undergone radical anterior resection or low anterior resection.

3. The food composition of claim 2, wherein the individual is an individual diagnosed with cancer.

4. The food composition of claim 3, wherein the cancer is rectal cancer or rectosigmoid colon cancer.

5. A health functional food for preventing or alleviating low anterior resection syndrome containing prune.
